(19) United States
(12) Patent Application Publication
(10) Pub. No.: US 2021/0065915 A1
(43) Pub. Date: Mar. 4, 2021

(54) INFORMATION PROCESSOR, INFORMATION PROCESING METHOD, AND RECORDING MEDIUM

(71) Applicant: SONY CORPORATION, TOKYO (JP)

(72) Inventor: HIROSHI IWANAMI, TOKYO (JP)

(21) Appl. No.: 16/960,776

(22) PCT Filed: Oct. 25, 2018
(86) PCT No.: PCT/JP2018/039745

§ 371 Date: Jul. 8, 2020

(30) Foreign Application Priority Data
Jan. 23, 2018 (JP) 2018-008608

(51) Int. Cl. G16H 50/80; G16H 40/67; G16H 50/30; G16H 15/00; G06Q 50/00; G06F 16/9038

(52) U.S. Cl. CPC G16H 50/80; G16H 40/67; G06F 16/9038; G16H 15/00; G06Q 50/01; G16H 50/30

(57) ABSTRACT

[Overview] [Problem to be Solved] To provide an information processor, an information processing method, and a recording medium that make it possible to automatically estimates a life rhythm of a community. [Solution] An information processor includes a control unit that performs a control. The control unit is configured to acquire sensor data obtained by sensing a member who belongs to a specific community, and automatically estimate a life rhythm of the member who belongs to the specific community, on the basis of the acquired sensor data.

### TECHNICAL FIELD

The present technology relates to an information processor, an information processing method, and a recording medium.

### BACKGROUND ART

In the world, there are some cases where members of a community therein naturally get into a unique behavior rhythm even though no rule is clearly defined. For example, in a family, even though there is no family percept or written rule, family members may gather for breakfast on holidays such as Saturdays, Sundays, etc. generally around 09:30. Around 09:30, all of the family members naturally begin to gather in a dining room and have breakfast, which then becomes a habit.

### CITATION LIST

### Patent Literature

PTL 1: International Publication No. WO2014/091766

PTL 2: Japanese Unexamined Patent Application Publication No. 2009-82263

### SUMMARY OF THE INVENTION

### Problems to be Solved by the Invention

Here it is believed that in a community such as a family, etc. there is something like a unique behavior rhythm thereof and that getting into that rhythm or avoiding that rhythm to the contrary results in various advantages. For example, mealtime of the family being taken as an example, a collateral advantage is expected that family members gathering in accordance with that rhythm obviously meet each other and have a talk, and therefore communications between the family members naturally improve. Such a tendency may occur in a group in which a community such as a family or a company is small to some extent, and differences arise between communities. However, in a recent mechanism of collecting all data such as big data, the differences between the communities are averaged and feature amounts of the communities get buried. Hence, it becomes valuable to focus on the small group community such as the family, collect not the big data but data therefrom, and analyze the tendency.

In the above-described PTL 1, as non-language information in communications between subjects, a nodding action, a body gesture, a hand gesture, movement of a body trunk, gaze stay time, voice tone, and a sigh are observed to evaluate a degree of tuning or synchronization thereof, which contributes to improvement of the communications. However, the PTL 1 does not mention automatically estimating a life rhythm of a community.

The above-described PTL 2 also relates to controlling light intensity on the basis of a biological rhythm that is judged on the basis of biological information acquired from a user of an electronic apparatus and adjusting the biological rhythm of the user of the electronic apparatus. Nevertheless, PTL2 does not mention the automatically estimating the life rhythm of the community.

Hence, the present disclosure proposes an information processor, an information processing method, and a recording medium that make it possible to automatically estimate a life rhythm of a community.

### Means for Solving the Problems

According to the present disclosure, there is proposed an information processor that includes a control unit that performs a control. The control unit is configured to acquire sensor data obtained by sensing a member who belongs to a specific community, and automatically estimate a life rhythm of the member who belongs to the specific community, on the basis of the acquired sensor data.

According to the present disclosure, there is proposed an information processing method that includes: acquiring, with a processor, sensor data obtained by sensing a member who belongs to a specific community; and automatically estimating, with the processor, a life rhythm of the member who belongs to the specific community, on the basis of the acquired sensor data.

According to the present disclosure, there is proposed a recording medium containing a program recorded therein that causes a computer to function as a control unit that performs a control. The control includes: acquiring sensor data obtained by sensing a member who belongs to a specific community; and automatically estimating a life rhythm of the member who belongs to the specific community, on the basis of the acquired sensor data.

### Effect of the Invention

As described above, according to the present disclosure, it is possible to automatically estimate a life rhythm of a community.

It is to be noted that the effects described here are not necessarily limiting, and any effect that is described herein or may be understood herefrom may be provided in addition to the above-described effects or in place of the above-described effects.

### BRIEF DESCRIPTION OF THE DRAWINGS

FIG. 1 is a diagram explaining about an overview of an information processing system according to an embodiment of the present disclosure.

FIG. 2 is a block diagram illustrating an example of the information processing system according to the embodiment of the present disclosure.

FIG. 3 is a flowchart illustrating basic action processing of the information processing system according to the embodiment of the present disclosure.

FIG. 4 is a block diagram illustrating an example of a specific configuration of an information processing system according to a first example.

FIG. 5 is a graph illustrating an example of a meal record according to the first example.

FIG. 6 is a diagram illustrating an example of a graph that represents life rhythms of a family according to the first example.

FIG. 7 is a flowchart illustrating action processing of generating a rhythm of dinner time according to the first example.

FIG. 8 is a diagram illustrating an example of a calculation expression of cumulative average time by a day of the week according to the first example.

FIG. 9 is a diagram illustrating a graph of a life rhythm of one day of a certain member of the family according to the first example.

FIG. 10 is a diagram illustrating an example of graphs each representing life rhythms by the day of the week of a certain member of a family according to a second example.

FIG. 11 is a flowchart illustrating visualization processing of the life rhythms of one week according to the second example.

FIG. 12 is a flowchart illustrating action processing of notification in a case of asynchronous life rhythms according to a third example.

FIG. 13 is a flowchart illustrating action processing on searched side in searching of another community according to a fourth example.

FIG. 14 is a flowchart illustrating action processing on searching side in the searching of the other community according to the fourth example.

### MODES FOR CARRYING OUT THE INVENTION

In the following, description is given in detail of a preferred embodiment of the present disclosure with reference to the accompanying drawings. It is to be noted that in this specification and the drawings, components having a substantially same functional configuration are denoted by same reference numerals, and overlapping description thereof is omitted.

In addition, description is given in the following order.

### 1. Overview of an Information Processing System According to an Embodiment of the Present Disclosure

### 2. Respective Examples

2-1. First Example

(2-1-1. Configuration Example)

(2-1-2. Action Processing)

2-2. Second Example

2-3. Third Example

2-4. Fourth Example

### 3. Conclusion

### 1. OVERVIEW OF INFORMATION PROCESSING SYSTEM ACCORDING TO EMBODIMENT OF PRESENT DISCLOSURE

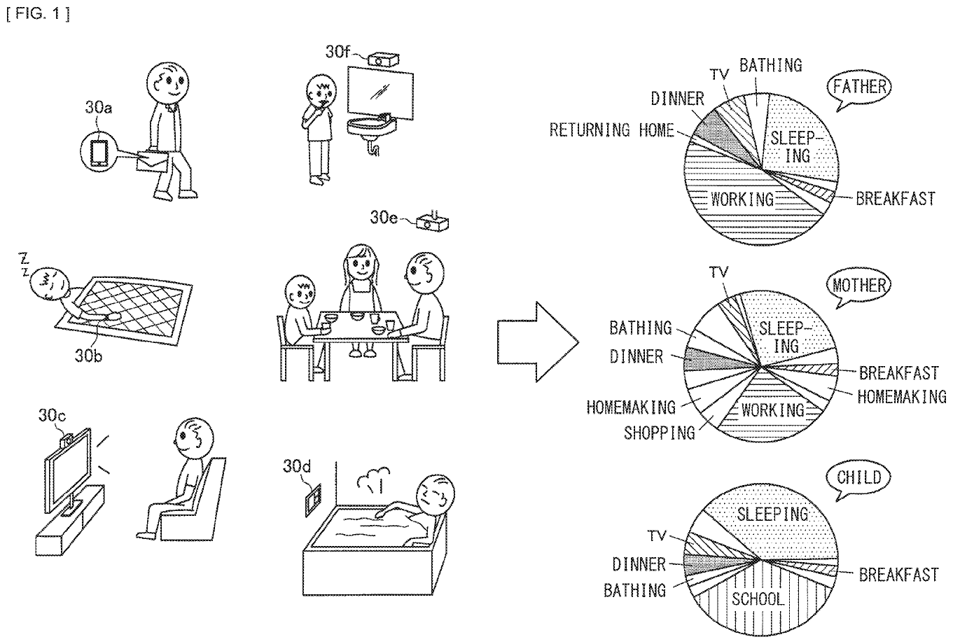

FIG. 1 is a diagram explaining about an overview of an information processing system according to an embodiment of the present disclosure. As illustrated in FIG. 1, in the information processing system according to the present embodiment, it is possible to focus on a small group community such as a family, a company, a school, or a neighborhood association, or the like and automatically estimate life rhythms of one day of respective members as illustrated on the right of FIG. 1, on the basis of data collected from the members (for example, family members) who belong to the community. A “life rhythm” herein means a cyclic living activity such as wake-up time, bedtime, mealtime, work time, exercise time, bath time, restroom time, media viewing time, and time spent happily by family members, or the like. In an example illustrated in FIG. 1, the automatically estimated life rhythms of one day (when and what kinds of activities take place) of the respective family members are visualized in pie charts, by way of example. It is also possible to automatically estimate a life rhythm of the family on the basis of the life rhythms of the respective members. Such a living activity is detected by various types of sensors 30, a camera or a microphone provided in a house, for example, a sensor system, and a mobile terminal. The camera is a camera 30c, a camera 30e, or a camera 30f as illustrated in FIG. 1. The microphone is hereinafter referred to as a mike. The sensor system is, for example, a hot-water supply panel 30d as illustrated in FIG. 1, and senses who is using a television set, a bathroom, a restroom, a kitchen, or the like, by means of face recognition, voice, an ID, fingerprint authentication, or the like. The mobile terminal is, for example, a smartphone (smartphone 30a as illustrated in FIG. 1) or a smart band (smart band 30b as illustrated in FIG. 1) possessed by each of the family members.

This system makes it possible to automatically estimate a Father's life rhythm, a Mother's life rhythm, and a Child's life rhythm, or the like, on the basis of data thus collected from the family and present the life rhythms to the family. This allows each of the family members to increase communications in the community or improve comfort of life by checking his or her life rhythm or that of another family member and synchronizing or intentionally not synchronizing his or her life rhythm with that of the other family member.

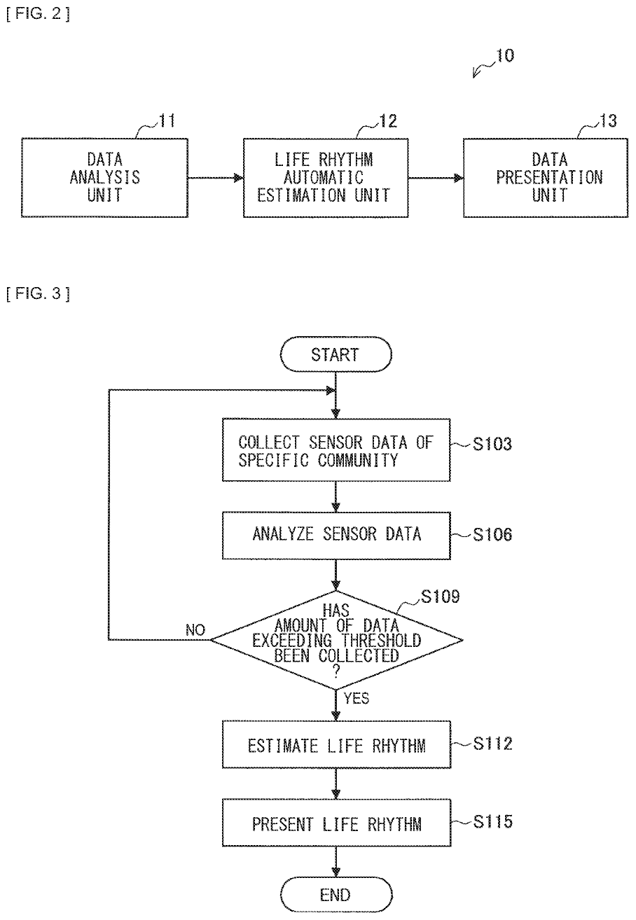

FIG. 2 illustrates a basic configuration of such an information processing system according to the embodiment. FIG. 2 is a block diagram illustrating an example of a system 10 according to the embodiment of the present disclosure. As illustrated in FIG. 2, the system 10 according to the present embodiment includes a data analysis unit 11, a life rhythm automatic estimation unit 12, and a data presentation unit 13. The system 10 may include a server on a network or may include a dedicated terminal such as a home agent or a client device such as the smartphone, a tablet terminal, or the like. The system 10 may also include a plurality of apparatuses.

The data analysis unit 11 analyzes sensing data resulting from sensing of activities of a member who belongs to a specific community such as a family.

The life rhythm automatic estimation unit 12 automatically estimates the life rhythm of the specific community on the basis of a result of the analysis of the data analysis unit 11. Here, as described above, the “life rhythm” means the cyclic living activity such as the wake-up time, the bedtime, the mealtime, the work time, the exercise time, the bath time, the restroom time, the media viewing time, and the time spent happily by the family members, communication time (such as talking, telephone calling, or conferencing), or the like. It is possible for the life rhythm automatic estimation unit 12 to estimate the life rhythms of respective members belonging to the specific community and to calculate the life rhythm that serves as a reference in the specific community, such as an average life rhythm of the specific community, on the basis of the life rhythms of the respective members.

The data presentation unit 13 performs control of presenting, as necessary, information regarding the life rhythm derived by the life rhythm automatic estimation unit 12 to another member of the community or members of another community. For example, the data presentation unit 13 may also graph and present the derived life rhythm.

FIG. 3 illustrates basic action processing of the system 10 having such a configuration. FIG. 3 is a flowchart illustrating the basic action processing of the information processing system according to the embodiment of the present disclosure.

As illustrated in FIG. 3, first, the system 10 collects sensor data of the specific community (step S103), and causes the data analysis unit 11 to analyze the sensor data (step S106).

Next, in a case where an amount of data that exceeds a threshold is gathered (step S109/Yes), the life rhythm automatic estimation unit 12 estimates the life rhythm of the specific community on the basis of the result of the analysis by the data analysis unit 11 (step S112).

Then, the data presentation unit 13 notifies the life rhythm of the specific community to community members, etc. (step S115). It is possible for the community members to effectively utilize the life rhythm, where appropriate, such as increasing communications, by knowing their life rhythms or those of the other members and thereby adapting their life rhythms to those of the other members.

As above, description has been given of the overview of the information processing system according to the embodiment of the present disclosure. In the following, specific description is given of the present embodiment, by means of a plurality of examples.

### 2. RESPECTIVE EXAMPLES

### 2-1. First Example

First, in a first example, description is given of an example of deriving a rhythm of dinner time as a family life rhythm.

### 2-1-1. Configuration Example

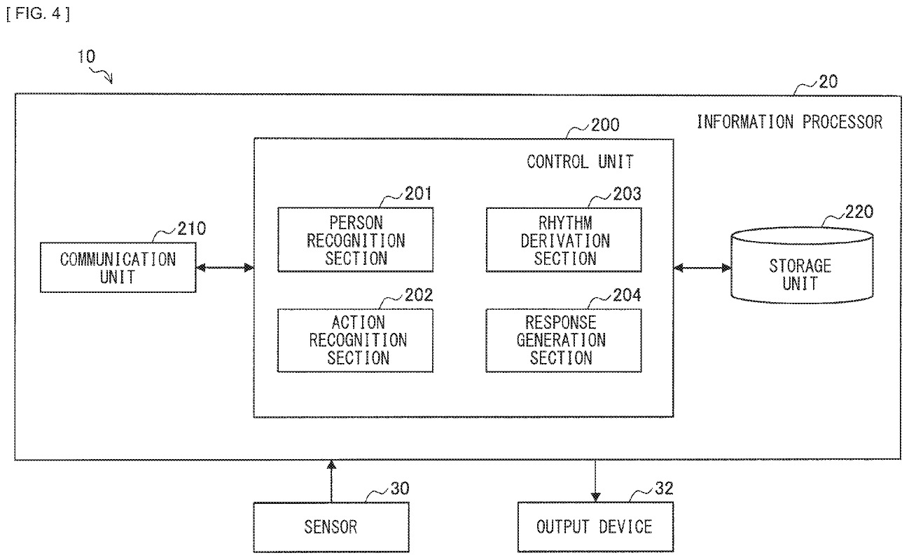

FIG. 4 is a block diagram illustrating an example of a specific configuration of the system 10 according to the first example. As illustrated in FIG. 4, the system 10 includes an information processor 20, the sensor 30 (or the sensor system), and an output device 32 (or an output system). The above-described data analysis unit 11, the life rhythm automatic estimation unit 12, and the data presentation unit 13 may be implemented by the information processor 20.

### (Sensor 30)

The sensor 30 is an apparatus/system that acquires all information related to a community member (user). For example, the sensor 30 may include a sensor on side of an environment, a motion sensor, and various types of sensors on side of the user. The sensor on the side of environment is a camera, a mike, or the like, provided in a room. The motion sensor is an acceleration sensor, a gyro sensor, a geomagnetic sensor, etc. provided on the smartphone or a wearable device possessed by the user. The various types of sensors on the side of the user are a biological sensor, a position sensor, a camera, and a mike, or the like. In addition, activity history of the user may be acquired over a network. The activity history is movement history, SNS, shopping history, or the like. The sensor 30 senses activities of members in a specific community on a daily basis and the information processor 20 collects sensed data.

### (Output Device 32)

The output device 32 is an expression device that notifies the members of the life rhythms or the like automatically estimated by the information processor 20. The output device 32 may broadly include IoT devices such as the smartphone, the tablet terminal, a portable phone terminal, a PC, a wearable device (HMD, smart eyeglass, the smart band, or the like), a TV set, lighting equipment, a speaker, or a vibration device.

### (Information Processor 20)

The information processor 20 includes a control unit 200, a communication unit 210, and a storage unit 220. The information processor 20 may include a cloud server on a network, may include an intermediate server or an edge server, may include the dedicated terminal placed in home, such as the home agent, or may include an information processing terminal such as the PC or the smartphone.

Control Unit 200

The control unit 200 functions as an arithmetic processing apparatus and a controller, and controls overall actions in the information processor 20 in accordance with various types of programs. The control unit 200 is implemented by an electronic circuit such as a CPU (Central Processing Unit) or a microprocessor, for example. In addition, the control unit 200 may include a ROM (Read Only Memory) and a RAM (Random Access Memory). The ROM stores a program or an operation parameter to be used, or the like. The RAM temporarily stores a parameter, or the like, that varies as appropriate.

In addition, the control unit 200 according to the present embodiment also functions as a person recognition section 201, an action recognition section 202, a rhythm derivation section 203, and a response generation section 204.

The person recognition section 201 performs person recognition by means of the face recognition of camera images, or the like. Alternatively, it is also possible for the person recognition section 201 to perform the person recognition by means of speaker recognition based on voice information or biometric authentication of a fingerprint, a vein, or the like.

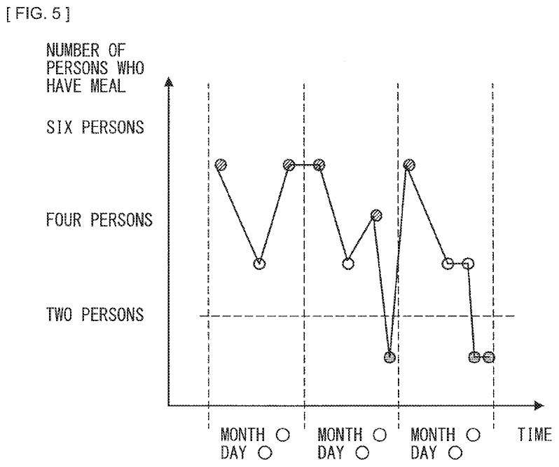

The action recognition section 202 recognizes an action of each user (living activity such as returning home or having a meal, taking bath, relaxing time, or retiring) on the basis of the camera images, the voice information, or various types of sensor data such as motion sensor data. That is, in order to estimate a life rhythm that is a rhythm of a predetermined living activity, the action recognition section 202 analyzes specific sensor data and outputs an analysis result to the rhythm derivation section 203. More specifically, for example, using taken images of a camera provided at an entrance, the action recognition section 202 may identify a person who returns home by means of the face recognition and record who returns and when. In addition, using taken images of a camera provided in a dining room, the action recognition section 202 may identify a person who is having a meal by means of the face recognition and record who has a meal and when. In addition, as illustrated in FIG. 5, the action recognition section 202 also takes a record of those who have a meal together, and the number of those persons, or the like.

It may be possible to recognize eating behavior by, for example, recognizing tableware, sensing chewing behavior from a relative position of a specific point in a face image, as well as an action of a person using chopsticks, a knife, a spoon, etc., and combining these. The chewing behavior is to move a jaw up and down. It is also possible to recognize the eating behavior from sound of the tableware being in contact with each other, chewing sound, and conversations, or the like, in addition to the taken images. It is to be noted that in this example, although description is given on the assumption that multiple sensors such as cameras or mikes are installed in a house to track conditions of the user, this example is not limited thereto. For example, in recent years, many people have a mobile terminal such as a smartphone on hand to continually check the SNS (Social Networking Service) or e-mails. Therefore, it is possible to keep track of many portions of the living activities (moving, having meals, sleeping, having conversations, or the like) of the user by using the camera or the mike of such mobile terminal.

The rhythm derivation section 203 derives, as the life rhythm, the life rhythms of the respective members of the family (for example, tendencies of the dinner time of the respective members by the day of the week, or the like) on the basis of the record of the living activities of the family. It is also possible for the rhythm derivation section 203 to derive, as the reference life rhythm, an average value of the life rhythms of the respective members, for example.

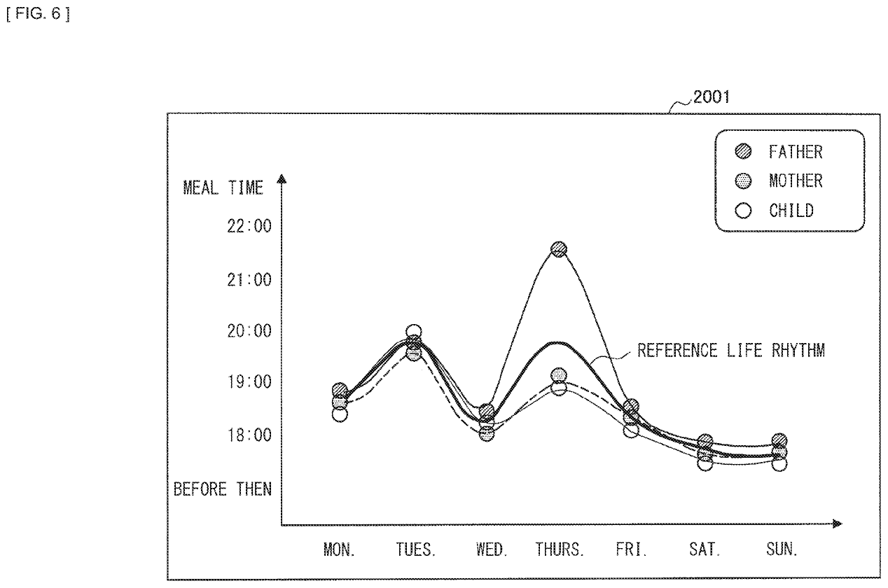

The response generation section 204 generates response information that outputs, as a result, the life rhythms of the respective members or the reference life rhythm of the family that are derived by the rhythm derivation section 203. For example, the response generation section 204 may generate a graph that represents the life rhythm by each of the members. Here, FIG. 6 illustrates an example of a graph that represents the life rhythms of the family that are automatically estimated on the basis of the collected data. In a graph 2001 illustrated in FIG. 6, as the life rhythms, the dinner time of Father, the dinner time of Mother, and the dinner time of Child, as well as dinner time (cumulative average time, for example) which serves as the reference of the family are depicted by the day of the week. For example, by checking such life rhythms, it is possible for Father to intuitionally understand that his mealtime widely deviates only on Thursdays. In addition, in a case where Father returns home late due to a regular meeting in a company on Thursdays and his dinner time is thus late, Father is expected to consider a possibility of shifting the regular meeting to Tuesdays when the dinner time of the family is relatively late, thereby being able to coincide his dinner time with that of the family.

The response information generated by the response generation section 204 is outputted from the output device 32 by the control unit 200 and notified to the family members. It is to be noted that a notice of the response information may also be conveyed by utilizing a message function via Internet such as the e-mails or the SNS. In addition, the response generation section 204 may directly or indirectly present an advice based on the life rhythm in addition to the notice of the life rhythm. Presentation of the advice may also be conveyed by utilizing the message function such as the e-mails or the SNS. In addition, although in an example illustrated in FIG. 6, the presentation of the life rhythm includes the tendencies by the day of the week, this example is not limited thereto.

### (Communication Unit 210)

The communication unit 210 couples to an external apparatus such as the sensor 30 or the output device 32, by wire or wirelessly, to perform transmission and reception of data. The communication unit 210 communicatively couples to the external device, for example, via a wired/wireless LAN (Local Area Network) or Wi-Fi (Registered trademark), Bluetooth (Registered trademark), a mobile communication network (LTE (Long Term Evolution) and 3G (Third-generation mobile telecommunications)), or the like.

### (Storage Unit 220)

The storage unit 220 is implemented by the ROM (Read Only Memory) and the RAM (Random Access Memory). The ROM stores the program or the operation parameter to be used in the processing of the control unit 200, or the like. The RAM temporarily stores the parameter that varies as appropriate.

As above, specific description has been given of the configuration of the system 10 according to this example. It is to be noted that the configuration of the system 10 is not limited to the example illustrated in FIG. 4. For example, the information processor 20 may include a plurality of apparatuses or may be integrated with the sensor 30 or the output device 32.

### 2-1-2. Action Processing

In the following, description is given of action processing of the system 10 according to this example with reference to a flowchart.

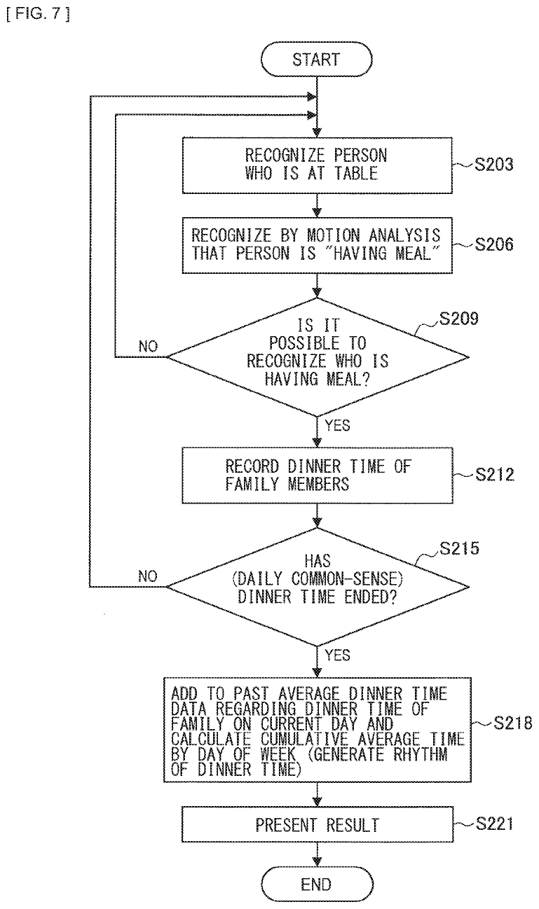

FIG. 7 is the flowchart illustrating the action processing of generating the rhythm of the dinner time according to this example. As illustrated in FIG. 7, first, the information processor 20 recognizes a person who is at a table by means of the camera or the mike, or the like (step S203), and recognizes that the person is “having a meal” by analysis (action analysis) of the camera images or the voice information (step S206). At this time, the information processor 20 further recognizes who is having a meal by the face recognition, or the like.

Next, in a case where it has been possible to recognize who is having a meal (all persons who are having a meal) (step S209/Yes), the information processor 20 records the dinner time of the family members (step S212). Here, the following table 1 lists an example of a record of the dinner time of the family members.

TABLE 1

| Person ID | Dinner Time |
|---|---|
| 00011 (Father) | Sep. 26, 2017 (Thurs.) 20:30 |
| 00012 (Mother) | Sep. 26, 2017 (Thurs.) 18:30 |
| 00013 (Child) | Sep. 26, 2017 (Thurs.) 17:30 |

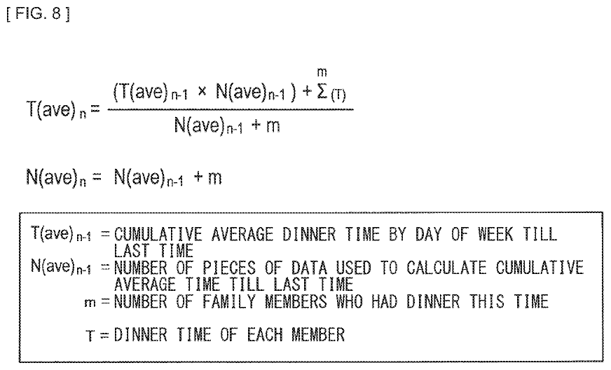

Then, in a case where (daily common-sense) dinner time ends (step S215/Yes), the information processor 20 adds data regarding the dinner time of the family of the current day to past average dinner time and calculates the cumulative average time by the day of the week (that is, generates the rhythm of the dinner time) (step S218). Here, FIG. 8 illustrates an example of a calculation expression of the cumulative average time by the day of the week. The information processor 20 may also calculate the cumulative average time by the day of the week, that is, the life rhythm, which serves as the reference of the family as illustrated in FIG. 6, by means of the calculation expression as illustrated in FIG. 8.

Then, the information processor 20 graphs the calculated life rhythms of the family as illustrated in FIG. 6 and presents it as a result (step S221).

### 2-2. Second Example

Although in the above-described first embodiment, description is given of the example of calculating the tendencies of the family dinner time as the life rhythm, it is also possible to calculate life rhythms other than the dinner time as described below.

Sleeping Time

When a user goes to bed and is in bed for a certain period, it is possible to measure sleeping time by a camera placed in a bed room, for example, on the basis of time when the user goes to bed and time when the user is out of bed. To improve accuracy, it is also acceptable not to count in the sleeping time a brief suspension such as getting up to go to the restroom, or the like. In addition, it is also possible to calculate the sleeping time by detecting heartbeat, breathing, or roll-over from vibration detection using the acceleration sensor/gyro sensor of the smartphone that the user brings to the bed and places by the bed. Moreover, in a case where the user wears a wearable terminal such as the smart band even while the user is sleeping, it is also possible to calculate the sleeping time of the user from the acceleration sensor/gyro sensor of the wearable terminal or a biosensor.

Restroom Time

It is possible to detect the restroom time by recognizing a person who enters the restroom or acquiring time of entry or exit with a camera placed in a hallway, for example. It is also possible to detect the restroom time by measuring a period of time during which the person sits on a toilet seat or identifying the user by weight, by means of a sensor incorporated in the toilet seat. In addition, it is also possible to identify users by means of the biosensor (fingerprint, etc.) provided on a door or a lighting switch of the restroom.

Leaving Home and Time to Return Home

It is possible to detect leaving home and time to return home by performing the facial recognition of those who enter or exit from the entrance by means of the camera installed at the entrance, for example.

Commuting Time

It is possible to identify from vibration of the acceleration sensor or the gyro sensor of the smartphone or the wearable terminal possessed by the user that the user is boarding a train, taking a ride in a car, walking, riding in an elevator, or the like. Therefore, it is possible to detect time commuting to work or a school on the basis thereof. In addition thereto, it is also possible to detect from a travel route the time commuting to work or the school by acquiring positional information of the smartphone or the wearable terminal.

Media Viewing Time

It is possible to detect media viewing time, such as watching television, by detecting that viewing of the television is performed or by performing identification of the user in front of a television set by means of a camera and a mike in a living room, for example.

Bath Time

It is possible to detect bath time by a camera placed at an entry of a bathroom, etc. However, it is expected that similarly to the restroom, placement of a camera is avoided due to consideration to privacy. In this case, it is possible to identify the user and detect the bath time from a rise of water level and an increased amount in the bathtub sensed by, for example, a water level sensor on a bathtub. In addition, it is also possible to detect the bath time by recognizing that user's hair is wet or that the user changes to night clothes or the like, on the basis of images of an in-camera of the smartphone that the user frequently utilizes or by recognizing sound from the user using a dryer or sound of shower, or the like, by means of the mike.

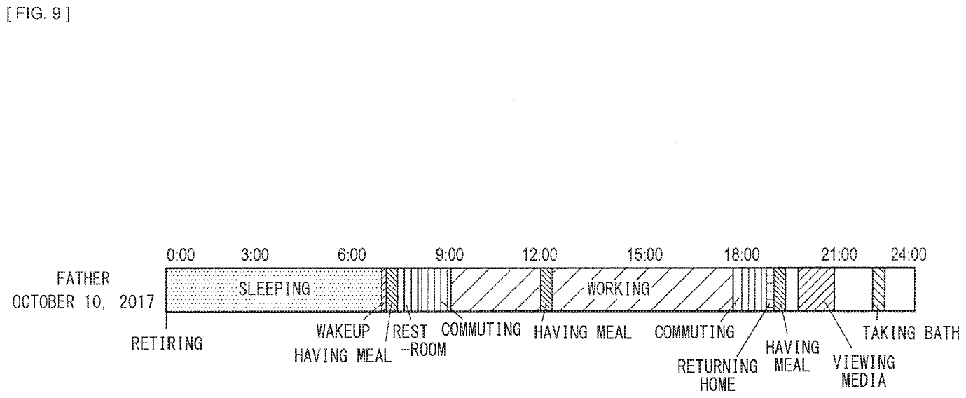

On the basis of the living activities of one day detected in this manner, it is possible to visualize the life rhythm of one day and notify it, as illustrated in FIG. 9. It is to be noted that in an example illustrated in FIG. 9, although the life rhythm of Father on a certain day is graphed, this example is not limited thereto. It is also possible to similarly graph the life rhythms of one day of the other family members.

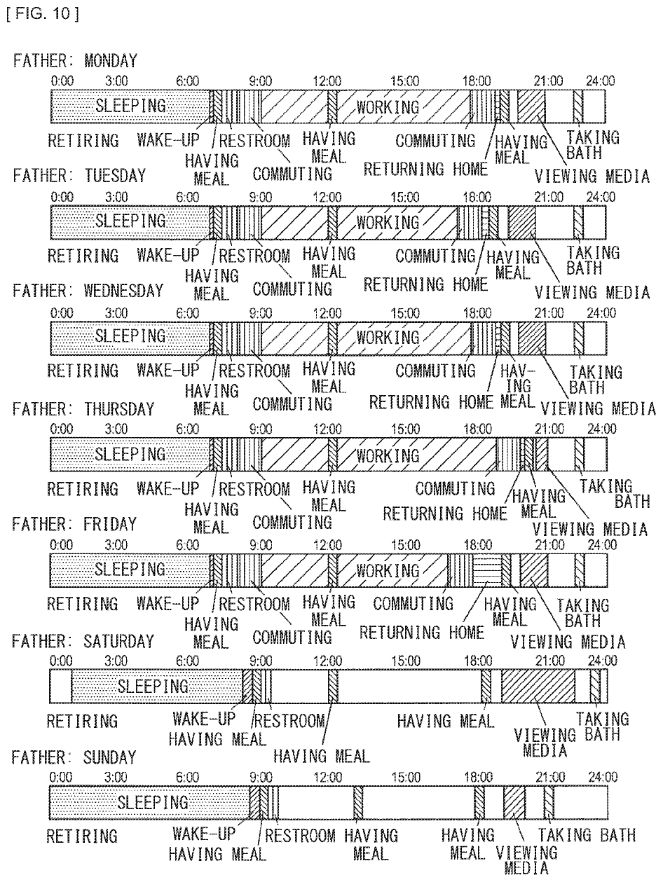

Moreover, it is possible to estimate and notify the life rhythms by the day of the week of the member, as illustrated in FIG. 10, by estimating and recording such life rhythms of one day for a long period of time, and averaging them by the day of the week.

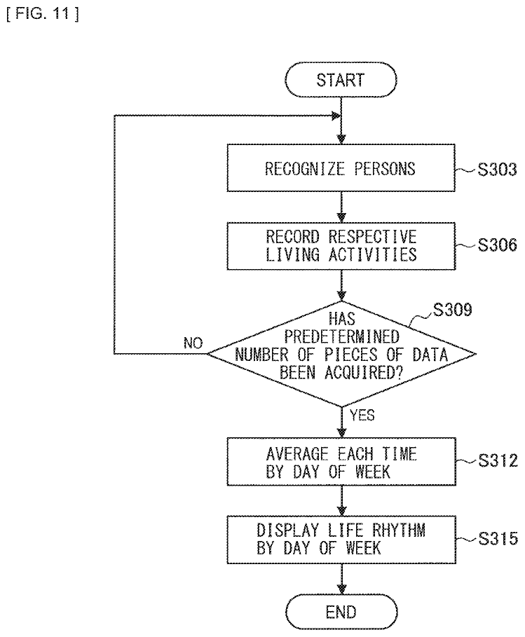

Here, FIG. 11 illustrates a flowchart of visualization processing of the life rhythms of one week. As illustrated in FIG. 11, first, the information processor 20 performs the recognition of a person (member) belonging to the specific community on the basis of the data sensed by the sensor 30 (step S303) and performs action recognition of the person to record each of the living activities (step S306).

Next, in a case where it is possible to acquire the predetermined number of pieces of data (step S309/Yes), the information processor 20 causes the response generation section 204 to average each time (time of each of the living activities) by the day of the week and generate (automatically estimate) the life rhythms of the member of one week (step S312).

Then, as illustrated in FIG. 10, for example, the response generation section 204 of the information processor 20 graphs the generated life rhythm by the day of the week and displays it on the output device 32 such as the smartphone of the member (step S315).

As above, description has been given of the visualization processing of the life rhythms of one week. It is to be noted that in the above-described example, the life rhythms of one week are graphed by determination of the average value of the time of the respective life rhythms (sleeping time, wake-up time, restroom time, mealtime, or the like), this example is not limited thereto and may be represented in a manner such as a heat map, for example. Moreover, it is possible to generate life rhythms of any period of time, such as life rhythms of one month, similarly to the calculation of the life rhythms of one week.

### 2-3. Third Example

Although in the above-described respective examples, description has been given of the cases of graphing and notifying the automatically estimated life rhythms, the present disclosure is not limited thereto. For example, it is also possible for the information processor 20 to notify the members in a case where the life rhythms enter a certain relationship in the community. This allows the members to improve their life rhythms in accordance with the notice, thereby increasing communications or realizing a more comfortable community.

### 2-3-1. Notifying in Case where Life Rhythms Become Out of Synchronization

In a case where the life rhythm of the member belonging to the specific community is out of the life rhythm that serves as the reference of the community, that is, is out of synchronization, for example, the information processor 20 may notify the member or all members. As illustrated in FIG. 6, for example, when the life rhythms of the dinner time of the family members are estimated, and in a case where the life rhythm of Father widely deviates from the life rhythm that serves as the reference of the family, the information processor 20 may notify Father that “Only your dinner time on Thursdays widely deviates”. Upon receipt of the notice, Father makes efforts to improve his life rhythm and, for example, attempts to synchronize with the life rhythm that serves as the reference of the family (rhythm of the dinner time). This increases a possibility that the family members have dinner together. That is, an effect is expected that the communications in the community increase.

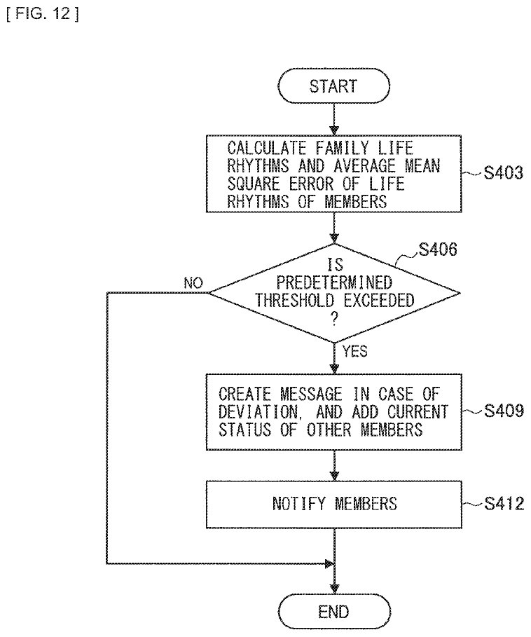

FIG. 12 is a flowchart illustrating action processing of notification in a case of asynchronous life rhythms according to this example. As illustrated in FIG. 12, first, the control unit 200 of the information processor 20 calculates a mean square error of the life rhythm of the family (that serves as the reference) and the life rhythms of the members (step S403). The life rhythm that serves as the reference of the family is, for example, the cumulative average time of the dinner time by the day of the week of the all family members.

Next, in a case where the calculated error exceeds a predetermined threshold (step S406/Yes), the control unit 200 causes the response generation section 204 to create a message in a case where the life rhythm deviates, and add a current status of the other members (step S409).

Then, the control unit 200 notifies the members of the created message (step S412). At this time, the control unit 200 may notify only the member who is out of synchronization or may notify all the members in the community. More specifically, for example, the control unit 200 gives a notice that “The family dinner time is out of rhythm” to all the family members. The control unit 200 may further inform Father, who is out of synchronization, of the current status of the other family members, such as “The family is now having a meal.” and may inform the family members other than Father of the current status of Father, such as “Father is still working.”

### 2-3-2. Notifying in Case where Life Rhythm is Synchronized

In contrast, in some cases, it is desirable that the life rhythms be asynchronous because congestion arises in a case where the life rhythms such as the restroom time, the bath time, or time for dressing in the morning, or the like, are synchronized. Hence, even in a case where specific life rhythms are synchronized, the control unit 200 may similarly notify the members accordingly.

For example, the information processor 20 derives bath time rhythms of the family members. In a case where a bath rhythm of Father approaches that of Mother, for example, the information processor 20 may give a notice informing them of synchronization. For example, the information processor 20 may output, from the output device 32 of a smartphone possessed by Father, a notice such as “Recently, your bath time is overlapping with that of Mother”. It is to be noted that the information processor 20 may notify at least someone of the members who are synchronized or another member who is out of synchronization.

### 2-4. Fourth Example

As described above, as life rhythms in each community become clear, it is possible to search a person in another community that meets a condition, on the basis of the life rhythms.

For example, a case is supposed in which a person who wishes to do running to lose weight after returning home from his or her company but has no one to run together in his or her family and feels sad to run alone. Furthermore, when it is not safe to run alone at late night, the person searches for a mate who could run with him or her in a same time zone, and they run together.

Alternatively, there is another case in which it is not quite easy for a person to get in touch with a business partner by phone. Then, the person wishing to know by any means when the business partner is in an office of a company searches a rhythm of the company of the business partner. The rhythm of the company includes, for example, time of a lunch break, around when employees engaged in work outside of the office return to the office, or the like.

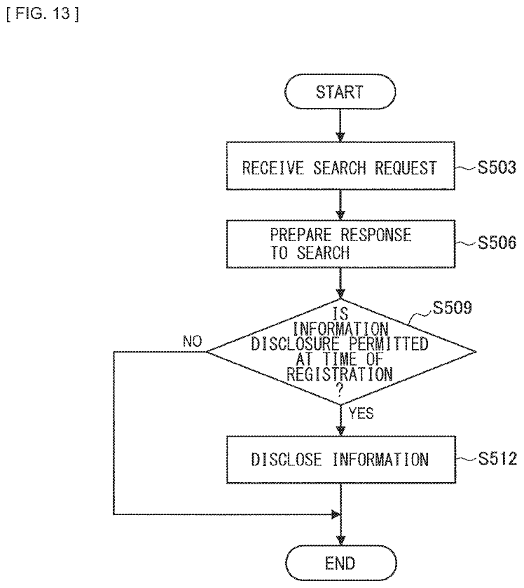

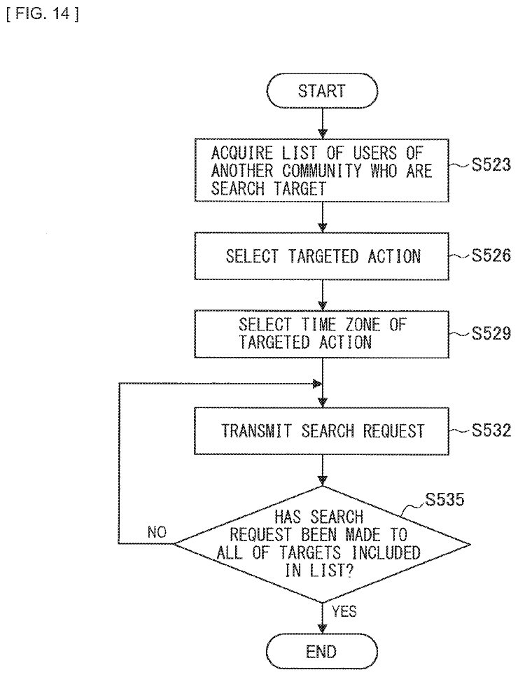

Here, in this example, search processing is enabled that makes life rhythms of members who permit disclosure a search target. In the following, description is given of the search processing according to this example with reference to FIG. 13 and FIG. 14.

FIG. 13 is a flowchart illustrating action processing on searched side in searching of another community according to this example. As illustrated in FIG. 13, when receiving a search request (step S503), the information processor 20 first prepares a response to a search (step S506). Here, by way of example, it is assumed that the search request has been made to the specific community by a member belonging to a different community. The information processor 20 searches the members belonging to the specific community for a member having a life rhythm that matches (or is similar to) the search request, for example, as preparation for the response to the search.

Next, the information processor 20 determines whether or not information disclosure has been permitted at the time of registration of the life rhythm (step S509). In this example, it is possible to register in advance whether or not each of the members of the community discloses his or her life rhythms to members of another community.

Then, in a case where the information disclosure is permitted (step S509/Yes), the information processor 20 performs the information disclosure (step S512). For example, the information processor 20 discloses the member who matches the search request or discloses the life rhythms of the member.

As above, description has been given of the case where the information processor 20 receives the search request. In the following, description is given of a case where the information processor 20 makes a search request with reference to FIG. 14. That is, description is given of a case where the member belonging to the specific community searches the other community.

FIG. 14 is a flowchart illustrating action processing on searching side in the searching of the other community according to this example. As illustrated in FIG. 14, the information processor 20 first acquires a list of users of the other community that is the search target (step S523). The list of the users of the other community may include a list of members who register with social media in which a certain member of the specific community, who is a searching person, for example, participates.

Next, the information processor 20 selects an action targeted for the search (step S526). For example, the member of the specific community (searching person) selects a living activity, such as running, that he or she wishes to search.

Then, the information processor 20 selects a time zone of the targeted action (step S529). For example, in a case where the member of the specific community (searching person) looks for a mate who does running at night, the searching person selects a specific time zone (for example, 22:00 to 25:00, or the like).

Next, the information processor 20 transmits the search request to targets listed in the acquired list of the users described above (step S532). The search request includes the targeted action and the time zone selected. It is to be noted that a search request is not limited to the request that specifies the targeted action and the time zone. A search request may be, for example, searching of a member having a similar life rhythm to a searching person himself or herself or searching of other community having a life rhythm similar to that of the community to which the searching person belongs, or the like.

Then, the information processor 20 makes the search request to all of the targets listed in the list of the users (step S535).

As above, description is given of the action processing on the search requesting side. It is to be noted that in an example illustrated in FIG. 14, although description has been given of the example of transmitting the search request, an example is conceivable in which searching is complete only in a server in a case where data such as the life rhythms of the respective members of the respective communities are all registered on the server.

### 3. CONCLUSION

As described above, in the information processing system according to the embodiment of the present disclosure, it is possible to automatically estimate a life rhythm of a community.

As above, description has been given in detail of the preferred embodiment of the present disclosure with reference to the accompanying drawings. However, the present technology is not limited to such examples. It is obviously possible that a person having ordinary skill in the art of the present disclosure conceive various kinds of alterations or modifications in a category of a technological idea described in the claims. It is to be understood that these alterations or modifications also naturally belong to the technological scope of the present disclosure.

For example, it is possible to create a computer program that causes hardware to exhibit capabilities of the information processor 20. The hardware includes the CPU, the ROM, and the RAM, or the like, that are built in the above-described information processor 20. In addition, a computer readable storage medium having the computer program stored therein is also provided.

In addition, the effects described herein are merely illustrative or exemplary, and not limiting. That is, a technique according to the present disclosure may have other effects that are apparent to those skilled in the art from the description herein, together with the above-described effects or in place of the above-described effects.

It is to be noted that it is possible for the present technology to take configurations as described below.

(1)

An information processor including

a control unit that performs a control, the control unit being configured to

acquire sensor data obtained by sensing a member who belongs to a specific community, and

automatically estimate a life rhythm of the member who belongs to the specific community, on the basis of the acquired sensor data.

(2)

The information processor according to (1), in which the control unit

analyzes specific sensor data to estimate the life rhythm that is a rhythm of a predetermined living activity, and

automatically estimates the life rhythm that is the rhythm of the predetermined living activity, in accordance with a result of the analysis.

(3)

The information processor according to (1) or (2), in which the control unit is configured to generate a graph that represents life rhythms of members, on the basis of the member belonging to the specific community.

(4)

The information processor according to (3), in which the control unit is configured to generate a graph that represents the life rhythms of one day, one week, or one month of the member.

(5)

The information processor according to any of (1) to (4), in which the control unit performs the control to cause a specific member to be notified when a relationship between life rhythms of members belonging to the specific community is in a certain situation.

(6)

The information processor according to (5), in which the control unit notifies a specific member of a plurality of members who belong to the specific community when a cycle of a specific living activity is out of synchronization for a certain length between the plurality of members.

(7)

The information processor according to (5), in which the control unit notifies a specific member who belongs to the specific community when specific living activities are substantially synchronized between a plurality of members who belong to the specific community.

(8)

The information processor according to any of (5) to (7), in which the control unit notifies at least either of a member for whom the relationship of the life rhythms is in the certain situation or another member, between the plurality of members who belong to the specific community.

(9)

The information processor according to any of (1) to (8) in which the control unit is configured to search a member who belongs to another community that is different from the specific community, on the basis of the life rhythms.

(10)

The information processor according to (9), in which the control unit presents only a member who permits disclosure as the result of the analysis.

(11)

The information processor according to (9) in which the control unit is configured to search a member having a similar life rhythm to a life rhythm of a specific member who belongs to the specific community, from members who are registered with social media in which the specific member participates, and to notify the specific member.

(12)

An information processing method including:

acquiring, with a processor, sensor data obtained by sensing a member who belongs to a specific community; and

automatically estimating, with the processor, a life rhythm of the member who belongs to the specific community, on the basis of the acquired sensor data.

(13)

A recording medium containing a program recorded therein that causes a computer to function as a control unit that performs a control, the control including:

acquiring sensor data obtained by sensing a member who belongs to a specific community; and

automatically estimating a life rhythm of the member who belongs to the specific community, on the basis of the acquired sensor data.

### REFERENCE NUMERAL LIST

10 System
20 Information processor
200 Control unit
201 Person recognition section
202 Action recognition section
203 Rhythm derivation section
204 Response generation section
210 Communication unit
220 Storage unit

1. An information processor, comprising:
a control unit configured to:
execute a control operation;
acquire sensor data obtained by sensing a member who belongs to a specific community; and
automatically estimate a life rhythm of the member based on the acquired sensor data.

2. The information processor according to claim 1, wherein the control unit is further configured to:
analyze specific sensor data to estimate the life rhythm that is a rhythm of a specific living activity, and
automatically estimate the life rhythm that is the rhythm of the specific living activity, based on a result of the analysis.

3. The information processor according to claim 1, wherein the control unit is further configured to generate a first graph that represents life rhythms of members, based on the member belonging to the specific community.

4. The information processor according to claim 3, wherein the control unit is further configured to generate a second graph that represents the life rhythms of one day, one week, or one month of the member.

5. The information processor according to claim 1, wherein the control unit is further configured to execute the control operation to cause a specific member of a plurality of members to be notified based on a relationship of life rhythms between members belonging to the specific community in a specific situation.

6. The information processor according to claim 5, wherein
the control unit is further configured to notify the specific member of the plurality of members who belong to the specific community, and
the notification is based on a cycle of a specific living activity that is out of synchronization for a certain length between the plurality of members.

7. The information processor according to claim 5, wherein
the control unit is further configured to notify the specific member who belongs to the specific community, and
the notification is based on specific living activities that are substantially synchronized among a plurality of members who belong to the specific community.

8. The information processor according to claim 5, wherein the control unit is further configured to notify at least one of a member of a plurality of members for whom the relationship of the life rhythms is in the specific situation or the other member of the plurality of members, among the plurality of members who belong to the specific community.

9. The information processor according to claim 1, wherein the control unit is further configured to search a member who belongs to a community that is different from the specific community, based on life rhythms.

10. The information processor according to claim 9, wherein the control unit is further configured to present a member who permits disclosure as a result of the search.

11. The information processor according to claim 9, wherein the control unit is further configured to:
search a member having a similar life rhythm to a life rhythm of a specific member who belongs to the specific community, from members who are registered with social media in which the specific member participates; and
notify the specific member.

12. An information processing method, comprising:
acquiring, with a processor, sensor data obtained by sensing a member who belongs to a specific community; and
automatically estimating, with the processor, a life rhythm of the member based on the acquired sensor data.

13. A non-transitory computer-readable medium having stored thereon, computer-executable instructions, which when executed by a processor of an information processing apparatus, cause the information processing apparatus to execute operations, the operations comprising:
acquiring sensor data obtained by sensing a member who belongs to a specific community; and
automatically estimating a life rhythm of the member based on the acquired sensor data.
